(19) United States
(12) Patent Application Publication
(10) Pub. No.: US 2016/0317127 A1
(43) Pub. Date: Nov. 3, 2016

(54) SMART DEVICE FOR ULTRASOUND IMAGING

(71) Applicant: QUALCOMM Incorporated, San Diego, CA (US)

(72) Inventors: Ricardo Paulo dos Santos Mendonca, Seattle, WA (US); Patrik Nils Lundqvist, Encinitas, CA (US); Rashid Ahmed Akbar Attar, San Diego, CA (US); Rajeev Jain, San Diego, CA (US); Padmapriya Jagannathan, San Diego, CA (US)

(21) Appl. No.: 15/140,006

(22) Filed: Apr. 27, 2016

Related U.S. Application Data
(60) Provisional application No. 62/153,978, filed on Apr. 28, 2015.
(60) Provisional application No. 62/153,970, filed on Apr. 28, 2015.
(60) Provisional application No. 62/153,974, filed on Apr. 28, 2015.

(51) Int. Cl. A61B 8/08; A61B 8/00

(52) U.S. Cl. CPC A61B 8/5215; A61B 8/467; A61B 8/4483; A61B 8/4444

(57) ABSTRACT

An ultrasonic imaging probe includes one or more ultrasonic transducers and one or more processors communicatively coupled with the one or more ultrasonic transducers. The one or more processors are configured to receive data from the one or more ultrasonic transducers and establish settings of the ultrasonic imaging probe based on the received data.

### CROSS-REFERENCE TO RELATED APPLICATIONS

This disclosure claims priority to U.S. Provisional Patent Application No. 62/153,978, filed on Apr. 28, 2015, entitled “AUTO-CONFIGURATION OF A DEVICE FOR ULTRASOUND IMAGING,” to Provisional Patent Application No. 62/153,970, filed on Apr. 28, 2015 and entitled “IN-DEVICE FUSION OF OPTICAL AND INERTIAL POSITIONAL TRACKING OF ULTRASOUND PROBES,” and to Provisional Patent Application No. 62/153,974, filed on Apr. 28, 2015 and entitled “OPTIMIZED ALLOCATION OF HETEROGENEOUS COMPUTATIONAL RESOURCES FOR ULTRASOUND IMAGING,” which are hereby incorporated by reference.

### TECHNICAL FIELD

This disclosure relates to an ultrasonic imaging probe, and more particularly to techniques for improving the operability and functionality of an ultrasonic imaging probe.

### DESCRIPTION OF THE RELATED TECHNOLOGY

High resolution ultrasonic imaging has been adapted for a large number of medical purposes. Traditionally, the ultrasonic imaging probe is a simple hand-held device that emits and receives acoustic signals. The device is connected by an electrical cable with a console or rack of equipment that provides control signals and power to the probe and that processes acoustic signal data received by the probe and forwarded to the console which processes the received data to produce viewable images of an anatomical feature of interest.

In the present disclosure, techniques are described for improving the operability and functionality of an ultrasonic imaging probe.

### SUMMARY

The systems, methods and devices of this disclosure each have several innovative aspects, no single one of which is solely responsible for the desirable attributes disclosed herein.

One innovative aspect of the subject matter described in this disclosure relates to an apparatus for ultrasonography. The apparatus includes one or more ultrasonic transducers and one or more processors communicatively coupled with the one or more ultrasonic transducers. The one or more processors are capable of receiving data from the one or more ultrasonic transducers and establishing settings for the apparatus based on the received data.

In some examples, the one or more processors may be configured to execute a process of establishing settings for the apparatus based on one or more machine learning processes, keyword detection processes or any combination thereof. In some examples, the settings for the apparatus may include any one or more of ultrasonic transducer frequency, ultrasonic transducer gain, signal processing filter parameters or any combination thereof. In some examples, at least one of the keyword detection processes may be responsive to an oral command. In some examples, the process of establishing settings for the apparatus includes one or both of initially setting up the apparatus and optimizing settings of the apparatus.

In some examples, the processor may include a trained machine learning engine and may establish settings for the apparatus based on outputs of the trained machine learning engine. In some examples, the trained machine learning engine may be configured to determine an organ (i), a type of examination (ii), a use case of a particular received image (iii), or any combination of (i), (ii) or (iii), and the outputs of the trained machine learning engine include presets of an ultrasonic transducer frequency (iv) an ultrasonic transducer gain (v), and signal processing filter parameters (vi), or any combination of (iv), (v) or (vi). In some examples, the trained machine learning engine may be configured to make a comparison of parameters from the received data to parameters established by a training data set and establish the settings based on the comparison.

In some examples, the one or more machine learning processes include training based on preferences expressed by an individual operator.

In some examples, at least one of the one or more processors may be a system on a chip that includes one of or more of a graphics processing unit (GPU), a digital signal processor (DSP), a central processing unit (CPU), a modem or any combination thereof. In some examples, the at least one processor may receive data from the one or more ultrasonic transducers and may be capable of generating an ultrasound image based on the received data. Generating the ultrasound image may include: (i) accessing a workflow, which includes one or more processing steps; (ii) assigning each processing step to one or more processing units, where the one or more processing units include one or more of: the GPU, the DSP, the CPU, the modem, another element of the ultrasonic imaging apparatus, or any combination thereof; (iii) generating one or more processed data at each of the one or more processing steps based on at least part of the received data; and (iv) generating an ultrasound image based on the one or more processed data. In some examples, assigning each processing step may be based on computational efficiency, power consumption metrics, an image quality metrics or any combination thereof.

According to some implementations, a method for ultrasonography includes receiving, with one or more processors, data from one or more ultrasonic transducers, the one or more processors and the one or more ultrasonic transducers being included in an apparatus for medical ultrasonography; and establishing settings for the apparatus based on the received data.

In some examples, the one or more processors may be configured to execute a process of establishing settings of the apparatus based on one or more machine learning processes, keyword detection processes, or any combination thereof. In some examples, the settings may include any one or more of ultrasonic transducer frequency, ultrasonic transducer gain, signal processing filter parameters, or any combination thereof. In some examples, at least one of the keyword detection processes is responsive to an oral command. In some examples, the process of establishing settings of the apparatus may include one or both of initially setting up the apparatus and optimizing settings of the apparatus.

In some examples, the processor may include a trained machine learning engine and establishes settings of the apparatus based on outputs of the trained machine learning engine. In some examples, the trained machine learning engine is configured to determine an organ (i), a type of examination (ii), a use case of a particular received image (iii), or any combination of (i), (ii) or (iii), and the outputs of the trained machine learning engine include presets of an ultrasonic transducer frequency (iv), an ultrasonic transducer gain (v), and signal processing filter parameters (vi), or any combination of (iv), (v) or (vi). In some examples, the trained machine learning engine may be configured to make a comparison of parameters from the received data to parameters established by a training data set and establish the settings based on the comparison.

In some examples, the machine learning process may include training based on preferences expressed by an individual operator.

According to some implementations, in a non-transitory computer readable medium having software stored thereon, the software includes instructions for causing an apparatus to: receive, with one or more processors, data from one or more ultrasonic transducers, the one or more processors and the one or more ultrasonic transducers being included in a portable ultrasonic apparatus for medical ultrasonography; and establish settings of the ultrasonic apparatus based on the received data.

### BRIEF DESCRIPTION OF THE DRAWINGS

Details of one or more implementations of the subject matter described in this specification are set forth in this disclosure and the accompanying drawings. Other features, aspects, and advantages will become apparent from a review of the disclosure. Note that the relative dimensions of the drawings and other diagrams of this disclosure may not be drawn to scale. The sizes, thicknesses, arrangements, materials, etc., shown and described in this disclosure are made only by way of example and should not be construed as limiting. Like reference numbers and designations in the various drawings indicate like elements.

FIG. 1 illustrates a hand-held ultrasonic imaging probe, according to an implementation.

FIG. 2 illustrates an example of a method for ultrasonography according to an implementation.

FIG. 3 illustrates an example of operation of a machine learning algorithm (“engine”), where it is assumed that the machine learning engine has already been trained.

FIG. 4 illustrates an example of a method of training the machine learning engine.

FIG. 5 illustrates a hand-held ultrasonic imaging probe, according to another implementation.

### DETAILED DESCRIPTION

Details of one or more implementations of the subject matter described in this specification are set forth in this disclosure, which includes the description and claims in this document, and the accompanying drawings. Other features, aspects and advantages will become apparent from a review of the disclosure. Note that the relative dimensions of the drawings and other diagrams of this disclosure may not be drawn to scale. The sizes, thicknesses, arrangements, materials, etc., shown and described in this disclosure are made only by way of example and should not be construed as limiting.

The present inventors have developed techniques for improving the portability, operability and functionality of ultrasonic scanners such that they may be used in a greater diversity of physical settings and by a user (care provider) who is not necessarily a specialized ultrasound technician (sonographer). For example, in a related provisional patent application entitled “AUTO-CONFIGURATION OF A DEVICE FOR ULTRASOUND IMAGING”, U.S. Provisional Patent Application No. 62/153,978, filed on Apr. 28, 2015, owned by the assignee of the present application, techniques are described for largely automating a process of setting up and/or optimizing settings of the ultrasonic probe. As a further example, in a related provisional patent application entitled, “OPTIMIZED ALLOCATION OF HETEROGENEOUS COMPUTATIONAL RESOURCES FOR ULTRASOUND IMAGING,” U.S. Provisional Patent Application No. 62/153,974, filed on Apr. 28, 2015, owned by the assignee of the present application, techniques are described for integrating, into a portable ultrasonic probe, a variety of specialized computational capabilities that conventionally would reside, if at all, in a separate console-type apparatus. As a yet further example, in a related provisional patent application entitled “IN-DEVICE FUSION OF OPTICAL AND INERTIAL POSITIONAL TRACKING OF ULTRASOUND PROBES”, U.S. Provisional Patent Application No. 62/153,970, filed on Apr. 28, 2015, owned by the assignee of the present application, techniques are described that enable a hand-held ultrasonic imaging probe to determine its own spatial position using optical and inertial sensors whether or not the probe is being used in a dedicated ultrasound examination room.

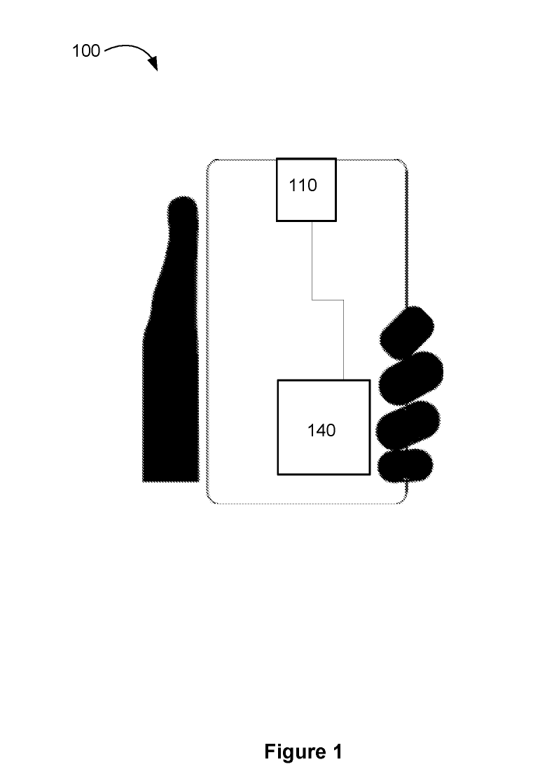

The systems, methods and devices of the disclosure each have several innovative aspects, no single one of which is solely responsible for the desirable attributes disclosed herein. One innovative aspect of the subject matter described in this disclosure can be implemented in an apparatus for medical ultrasonography. In some implementations, the apparatus may be a portable ultrasonic imaging probe. In some implementations, the portable ultrasonic imaging probe may be configured as a hand-held device. In some implementations the apparatus may be included in or attached to an apparatus such as a robot, or may be or include a wearable device. FIG. 1 illustrates a hand-held ultrasonic imaging probe, according to an implementation. The apparatus 100 includes an ultrasonic transducer 110 and a processor 140 communicatively coupled with the ultrasonic transducer 110.

In some implementations, the processor is configured to automate a process of establishing settings of the ultrasonic imaging probe on the basis of one or both of a machine learning process and keyword detection. The processor may be or include a system-on-a-chip including a robust set of heterogeneous computational elements such as the Snapdragon™ processor developed by the assignee of the present invention.

In the absence of the presently disclosed techniques, configuring an ultrasonic scan system for best quality imaging is a complicated process normally executable only by a specially trained sonographer.

In accordance with the presently disclosed techniques, several aspects of the process of configuring the ultrasonic scan system are substantially automated. As a result, ultrasonic scan technology may be made accessible to less highly trained care providers operating in a broader range of clinical or nonclinical settings such as an emergency room, an office of a primary care physician, in a patient's home, in athletic training facilities, in physical therapy facilities, and the like.

In an implementation, the processor of the ultrasonic imaging probe may be programmed to assist a user in configuring the ultrasonic imaging probe. For example, the processor may be capable of receiving data from one or more ultrasonic transducers and establishing (setting up, adjusting and/or optimizing) parameters (“settings”) based on the received data. More particularly, the received data, corresponding to an ultrasound image, may be analyzed and used by the processor to establish values for the settings so as to optimize image quality, and/or simplify the normally complicated process of preparing for an ultrasonic scan.

In an implementation, the processor of the ultrasonic imaging probe may be programmed to assist a user in configuring the ultrasonic imaging probe by automating a process of establishing settings of the ultrasonic probe on the basis of one or both of a machine learning process and keyword detection.

“Machine learning” (supervised or unsupervised), as the term is used herein and in the claims, refers to aspects of computational intelligence, that may include association rule learning, support vector machine (SVM) models, Random Forests (RF), deep learning (DL), evolutionary computation systems, and artificial neural networks, among others. Association rule learning relates to discovery of relations between variables. In the context of the present disclosure, association rule learning may include, for example, determining a relationship between an examination parameter and a probe setting.

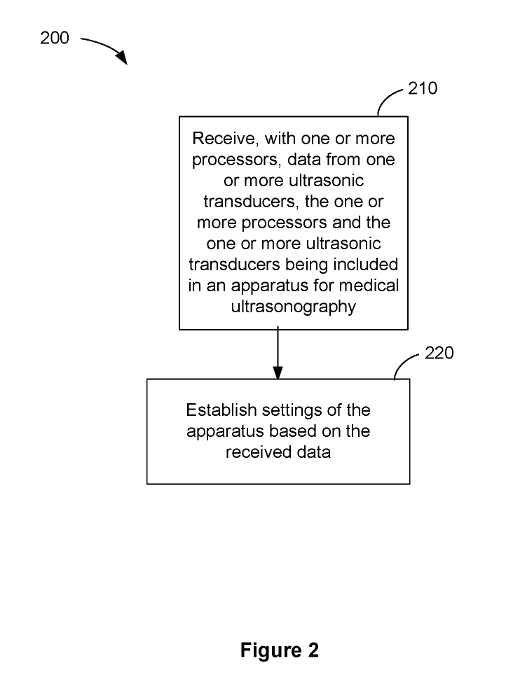

FIG. 2 illustrates an example of a method for ultrasonography according to an implementation. As described hereinabove, the ultrasonography apparatus may be configured as a portable ultrasonic imaging probe or device that includes one or more ultrasonic transducers and one or more processors communicatively coupled with the ultrasonic transducer(s). In the illustrated implementation, method 200 includes a block 210 for receiving, with the one or more processors, data from the one or more ultrasonic transducers.

The method proceeds, at block 220, with establishing settings of the apparatus based on the received data. For example, the processor may use analyze the received data, corresponding to an ultrasound image, so as to determine and/or establish values for the settings so as to optimize image quality, and/or simplify the normally complicated process of preparing for an ultrasonic scan

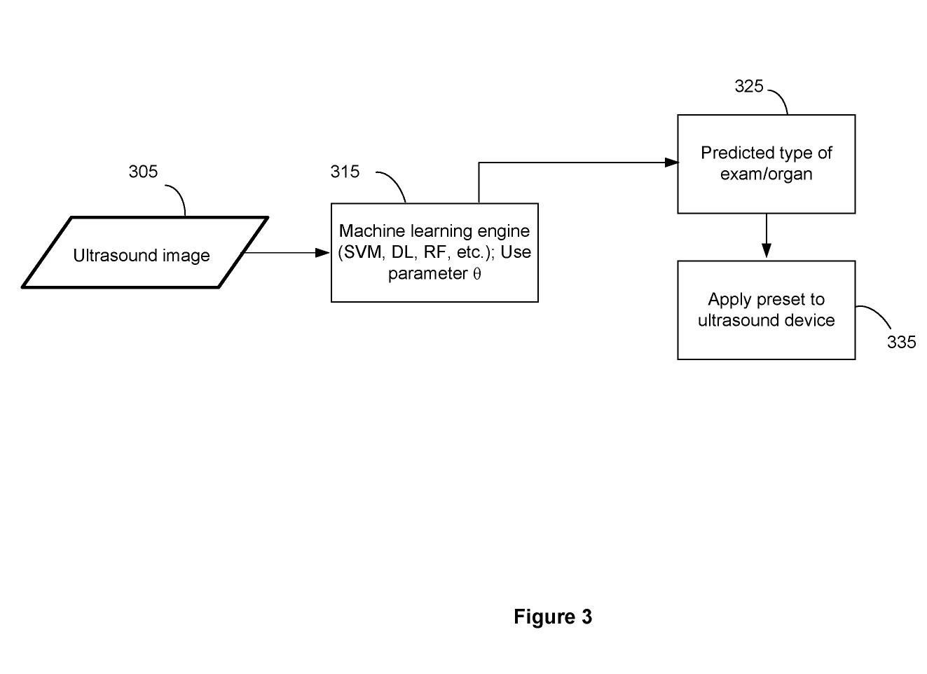

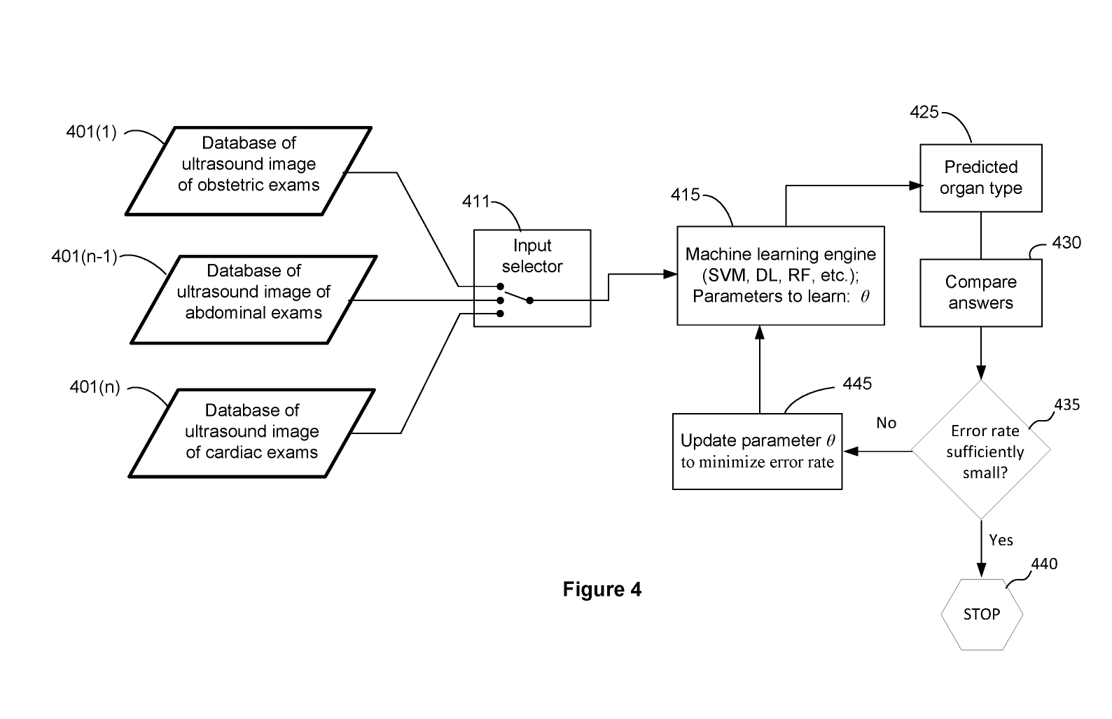

In an implementation, the processor may be configured to be initially set up and/or optimize settings of the ultrasonic probe in an autonomous or semi-autonomous mode based on a comparison of a received ultrasound image with a database of previously obtained ultrasound images, using machine learning techniques. FIG. 3 illustrates an example of operation of a machine learning algorithm (“engine”), where it is assumed that the machine learning engine has already been trained. FIG. 4 illustrates an example of a method of training the machine learning engine.

Referring to FIG. 3, an initially received ultrasound image, block 305, which may be obtained using an initial default set of operating parameters, is forwarded to the trained machine learning engine, block 315. The trained machine learning engine may be configured to recognize, from the initially received ultrasound image, the type of examination being performed and/or features of the tissue or anatomical feature that is being scanned. The trained machine learning engine may be configured to recognize a plurality of characteristic relationship parameters θ in the initially received ultrasound image sufficient to identify the image as representative of, for example, an obstetric exam, an abdominal exam, or a cardiac exam. For example, the machine learning engine may have been trained, using a sufficiently large data set, to associate features of an image with a particular organ type, so that when presented with a novel, unlabeled ultrasound image, the machine learning engine will correctly identify the imaged organ. In some implementations, keypoints in the ultrasound image and/or associated volume may be identified and compared to a model, the model having been preprogrammed or developed via a training set of data.

Based on a resulting prediction of the type of examination/organ being examined, block 325, the trained machine learning engine may apply, at block 335, appropriate presets to the ultrasonic imaging probe. For example, where the prediction at block 325 is that the examination is a cardiac exam, a frame rate of the ultrasonic scanner may be set relatively high so as to obtain blood flow rate information and a penetration depth may be set relatively deep (by, for example lowering the operating frequency of the ultrasonic transducer). As a further example, where the prediction at block 325 is that the examination is an abdominal exam, a frame rate of the ultrasonic scanner may be set relatively low.

Referring to FIG. 4, an example of techniques for training the machine learning engine is illustrated. A plurality of ultrasonic image databases 401(i) may be used for the training, each database being associated with a particular examination and/or organ type. For example in the simplified illustration to FIG. 4, the database 401(1) relates to obstetric exams, the database 401(n−1) relates to abdominal exams, and a database 401(n) relates to cardiac exams.

Images from the respective databases are selected, at block 411, and forwarded to the machine learning engine 415. An objective of the training may be to develop a sheaf of characteristic relationship parameters θ that optimally correlates characteristics of the ultrasound images to an examination type. The characteristic relationship parameters θ may be optimized by iteratively forming, for each image in a sufficiently large database of images of each particular type, a prediction, at block 425, of a type of examination/organ associated the image. At block 430, the prediction may be compared to the actual type of examination/organ represented by the image. At block 435 a determination may be made as to whether an error rate is sufficiently small (i.e., the machine learning engine meets a criteria for accuracy). If the determination at block 435 is that the error rate is sufficiently small in the method may stop, block 440. On the other hand, if the determination at block 435 is that the error rate is not sufficiently small results of the comparison at block 430 may be used to update, block 445, one or more of the characteristic relationship parameters θ with an objective, reducing the error rate.

As a result of such training, the processor of the ultrasonic imaging probe may be configured to determine the type of examination/organ and/or use case (cardiology, fetal monitoring, carotid artery monitoring, etc.) relating to a particular received image. The processor may be further configured to establish or adjust settings of the ultrasonic imaging transducer so as to produce a better image. The settings that may be established or adjusted may include, for example, contrast, brightness, frequency, frame rate, time-gain control, field of view, depth, beamforming settings (apodization, and delays, shape, and amplitude of each voltage pulse sent to each transducer) and signal processing filter parameters.

Alternatively, or in addition, the processor may be configured to establish the settings based on operator keyword inputs. In some implementations, the keyword inputs may be made orally and the processor may include voice recognition software responsive to the oral input. For example, an operator may use an oral command to identify use-case/organ e.g., ‘heart’, ‘fetus’, ‘thyroid’ etc. and the processor of the ultrasonic imaging probe may set, responsive to the keyword, ultrasonic transducer frequency and gain, signal processing filter parameters, etc., so as to produce a better image.

Similarly, the processor may be configured to respond to operator instructions to ‘increase/decrease’ contrast/brightness' add additional capabilities such as Doppler to Ultrasound imaging in Cardiology e.g., ‘add/remove Doppler’.

In an implementation, the machine learning techniques and keyword detection techniques described above may be combined. As a result, for example, an initial default configuration for an ultrasonic examination may be based on keyword detection. Subsequently, during the ultrasonic examination the machine learning algorithm may analyze a sequence of images captured by the ultrasonic imaging probe to further enhance the image quality by fine-tuning of frequency, gain, brightness, contrast etc. Implementation of the machine learning techniques and/or the keyword detection techniques may facilitate use of the ultrasonic imaging probe by non-experts. Moreover, the use of keyword detection may result in a better workflow that does not require the user to shift focus from the patient and/or ultrasound images to a control console in order to make adjustments in ultrasound parameters.

In some implementations, the machine learning algorithm may be configured to determine a correlation between input images and preferred probe settings including, for example, contrast, brightness, frequency, frame rate, time-gain control, field of view, depth, beamforming settings and/or signal processing filter parameters. For example, the machine learning algorithm may be trained by a training set including a number of image-to-new-setting pairs, where the images in the pairs, already classified as to a respective imaged organ, were still sub-optimal images acquired with default settings, and the new settings are those obtained from the default values after fine tuning of probe settings by an expert. As a result, the machine learning algorithm may be trained to associate input images to probe setting values in a continuous space (frame rate, frequency, brightness contrast, etc.).

This procedure could be done online, after the system is deployed, by using, as image-settings training pairs, the initial images that the system produces and the final setting after expert adjustments. Over a period of time, expert adjustments would become progressively less necessary. Alternatively, or in addition, some or all of the training could be done offline. Where at least some of the training is performed online, the disclosed techniques may allow for personalization, because the machine learning algorithm may be configured to learn the preferred settings of a specific user, in addition to or instead of generic settings of derived from an offline training set.

In some implementations a portable apparatus for medical ultrasonography includes a processor that is or includes a system-on-a-chip. The system-on-a-chip includes a diverse heterogeneous set of specialized computational elements such as DSPs, GPUs, CPUs and/or modems. The processor may include any combination of discrete components and/or combination chipset components, such as system-on-a-chip. For example, the processor may include a discrete GPU with a combination chipset that includes a DSP, CPU and a modem. In another example, the processor may include a combo chip that include a GPU, CPU, DSP and a Wide Area Network modem and a another combo chip that includes a Wireless Local Area Network modem, Bluetooth modem and Global Navigation Satellite System modem. The specialized computational elements may be individually matched to the computational requirements of specific algorithmic blocks of an ultrasound image acquisition and data processing workflow.

In the absence of the presently disclosed techniques, the low power requirements of portable or handheld ultrasound probes lead to a compromised image quality as a result of difficult trade-offs between power consumption and image quality. Conventional ultrasound probes, to the extent they incorporate data processing capabilities at all, generally rely on a single computational element—for example, a central processing unit (CPU), a digital signal processor (DSP), a graphical processing unit (GPU) or a field programmable gate array (FPGA)—to execute all algorithmic blocks in the acquisition and processing of ultrasound images. As a result, computational inefficiencies arise that result in either or both of a further increase in power consumption or a reduction in image quality.

According to one aspect of the presently disclosed techniques, different processing steps in a workflow of receiving and processing ultrasound image data may be allocated to different computational elements of the heterogeneous set of elements. Such allocation may include, for example, mapping each processing step in the workflow to the computational element best equipped to handle it, while considering the cost of moving data between different elements.

For example, the present disclosure contemplates that traditional signal processing tasks, such as low pass, band pass, or high pass filtering may be allocated to a DSP, whereas warping of the ultrasound image from a probe coordinate system to a coordinate system of a display would be allocated to a GPU. In an implementation, the DSP may be or include a Hexagon™ (QDSP6), developed by the assignee of the present invention, or similar device. In an implementation, the GPU may be or include an Adreno™ GPU, developed by the assignee of the present invention. At the same time, the computation of transmit and receive delays could be efficiently carried out by the CPU. Each of these computational elements, advantageously, may be integrated into a system-on-a-chip (SOC) that is incorporated within the portable device, as opposed to a separate equipment rack or console.

In an implementation, processed data may be generated at each of the one or more processing steps based on at least part of the received ultrasound image data and an ultrasound image may be generated based on the processed data.

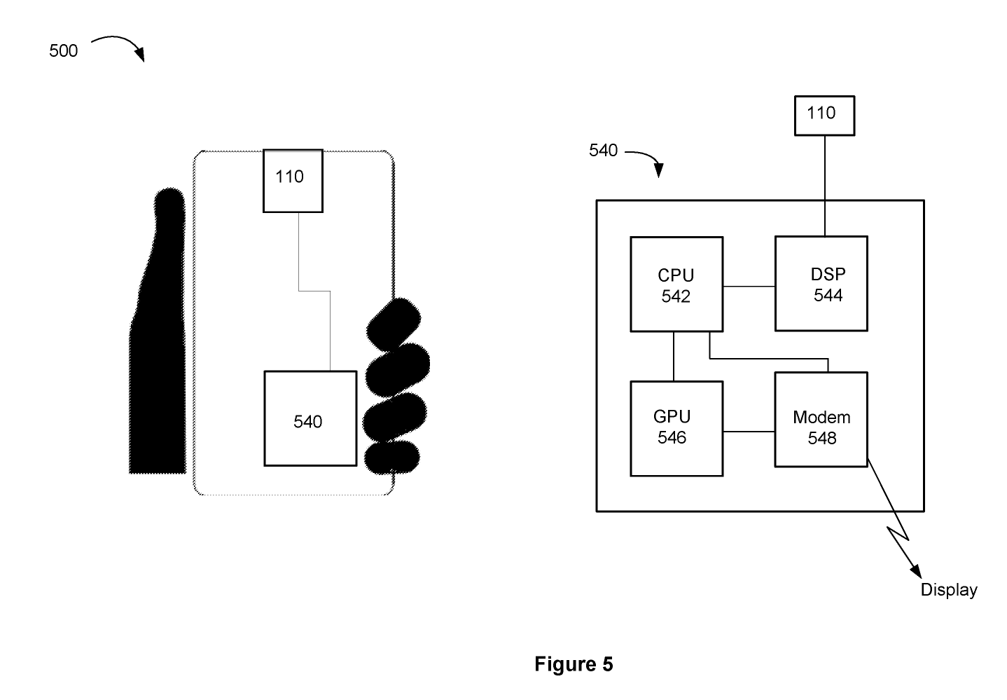

FIG. 5 illustrates a hand-held ultrasonic imaging probe, according to another implementation. The apparatus 500 includes an ultrasonic transducer 110 and a processor arrangement 540 communicatively coupled with the ultrasonic transducer 110.

The processor arrangement 540 may be configured as a SOC processor that includes a diverse heterogeneous set of specialized elements. In the illustrated implementation, for example the processor arrangement 540 includes a CPU 542, a DSP 544, a GPU 546 and a modem 548. The processor arrangement 540, whether or not configured as a SOC, may be configured to control and process ultrasound image data from the ultrasonic transducer 110, using appropriate ones of the specialized elements. For example, the DSP 544 may be primarily relied upon for interfacing with the ultrasonic transducer 110 and for performing functions such as low pass, band pass, or high pass filtering. As a further example, the GPU 546 may be primarily responsible for translating ultrasound image data defined in terms of the probe coordinate system to image data defined in terms of the display coordinate system. As a yet further example, the modem 548 may be primarily relied upon for communication with a separate display (not illustrated). As a result of such mapping of each processing step onto at least one of the CPU 542, the DSP 544, the GPU 546 and the modem 548 computational inefficiencies may be reduced and a power consumption metric and/or an image quality metric of the ultrasonic imaging probe may be improved.

The modem may communicate with the separate display wirelessly, or by way of a wired interface. For example, the modem 548 and the separate display may be coupled with each other and/or with a wide area network access point by way of a wireless local area network, a personal area network, or a piconet.

A wireless link between the modem 548 and the separate display may conform to the Bluetooth or other personal area network wireless communication standard. In some implementations, the wireless link may conform to one or more of the IEEE 802.11 (“WLAN”) standards, or another wireless standard suitable for use in a local area network, a personal area network, or a piconet.

Thus, a smart device for ultrasound imaging has been disclosed that may be configured as a portable or hand-held ultrasonic imaging probe and may include one or more processors configured to receive data from one or more ultrasonic transducers and establish settings of the ultrasonic imaging probe based on the received data.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor or any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by or to control the operation of data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that can be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, as a person having ordinary skill in the art will readily appreciate, the terms “upper” and “lower”, “top” and bottom”, “front” and “back”, and “over”, “on”, “under” and “underlying” are sometimes used for ease of describing the figures and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

What is claimed is:

1. An apparatus for ultrasonography, the apparatus comprising:
one or more ultrasonic transducers; and
one or more processors communicatively coupled with the one or more ultrasonic transducers, wherein the one or more processors are capable of:
receiving data from the one or more ultrasonic transducers; and
establishing settings for the apparatus based on the received data.

2. The apparatus of claim 1, wherein the one or more processors are configured to execute a process of establishing settings for the apparatus based on one or more machine learning processes, keyword detection processes or any combination thereof.

3. The apparatus of claim 2, wherein the settings for the apparatus include any one or more of ultrasonic transducer frequency, ultrasonic transducer gain, signal processing filter parameters or any combination thereof.

4. The apparatus of claim 2, wherein at least one of the keyword detection processes is responsive to an oral command.

5. The apparatus of claim 2, wherein the process of establishing settings for the apparatus includes one or both of initially setting up the apparatus and optimizing settings of the apparatus.

6. The apparatus of claim 2, wherein the processor includes a trained machine learning engine and establishes settings for the apparatus based on outputs of the trained machine learning engine.

7. The apparatus of claim 6, wherein the trained machine learning engine is configured to determine an organ (i), a type of examination (ii), a use case of a particular received image (iii), or any combination of (i), (ii) or (iii), and the outputs of the trained machine learning engine include presets of an ultrasonic transducer frequency (iv) an ultrasonic transducer gain (v), and signal processing filter parameters (vi), or any combination of (iv), (v) or (vi).

8. The apparatus of claim 6, wherein the trained machine learning engine is configured to make a comparison of parameters from the received data to parameters established by a training data set and establish the settings based on the comparison.

9. The apparatus of claim 2, wherein the one or more machine learning processes include training based on preferences expressed by an individual operator.

10. The apparatus of claim 1, wherein at least one of the one or more processors is a system on a chip that includes one of or more of a graphics processing unit (GPU), a digital signal processor (DSP), a central processing unit (CPU), a modem or any combination thereof.

11. The apparatus of claim 10, wherein:
the at least one processor receives data from the one or more ultrasonic transducers and is capable of:
generating an ultrasound image based on the received data, wherein the generating the ultrasound image comprises:
accessing a workflow, which comprises one or more processing steps;
assigning each processing step to one or more processing units, wherein the one or more processing units comprise one or more of: the GPU, the DSP, the CPU, the modem, another element of the apparatus, or any combination thereof;
generating one or more processed data at each of the one or more processing steps based on at least part of the received data; and
generating an ultrasound image based on the one or more processed data.

12. The apparatus of claim 11, wherein assigning each processing step is based on computational efficiency, power consumption metrics, an image quality metrics or any combination thereof.

13. A method for ultrasonography, the method comprising:
receiving, with one or more processors, data from one or more ultrasonic transducers, the one or more processors and the one or more ultrasonic transducers being included in an apparatus; and
establishing settings for the apparatus based on the received data.

14. The method of claim 13, wherein the one or more processors are configured to execute a process of establishing settings of the apparatus based on one or more machine learning processes, keyword detection processes, or any combination thereof.

15. The method of claim 14, wherein the settings include any one or more of ultrasonic transducer frequency, ultrasonic transducer gain, signal processing filter parameters, or any combination thereof.

16. The method of claim 14, wherein at least one of the keyword detection processes is responsive to an oral command.

17. The method of claim 14, wherein the process of establishing settings of the apparatus includes one or both of initially setting up the apparatus and optimizing settings of the apparatus.

18. The method of claim 14, wherein the processor includes a trained machine learning engine and establishes settings of the apparatus based on outputs of the trained machine learning engine.

19. The method of claim 18, wherein the trained machine learning engine is configured to determine an organ (i), a type of examination (ii), a use case of a particular received image (iii), or any combination of (i), (ii) or (iii), and the outputs of the trained machine learning engine include presets of an ultrasonic transducer frequency (iv), an ultrasonic transducer gain (v), and signal processing filter parameters (vi), or any combination of (iv), (v) or (vi).

20. The method of claim 18, wherein the trained machine learning engine is configured to make a comparison of parameters from the received data to parameters established by a training data set and establish the settings based on the comparison.

21. The method of claim 14, wherein the machine learning process includes training based on preferences expressed by an individual operator.

22. A non-transitory computer readable medium having software stored thereon, the software including instructions for causing an apparatus to:
receive, with one or more processors, data from one or more ultrasonic transducers, the one or more processors and the one or more ultrasonic transducers being included in an ultrasonic apparatus; and
establish settings of the ultrasonic apparatus based on the received data.

23. The computer readable medium of claim 22, wherein the one or more processors are configured to execute a process of establishing settings of the apparatus based on one or more machine learning processes, keyword detection processes or any combination thereof.

24. The computer readable medium of claim 23, wherein the settings include any one or more of ultrasonic transducer frequency, ultrasonic transducer gain, signal processing filter parameters or any combination thereof.

25. The computer readable medium of claim 23, wherein at least one of the keyword detection processes is responsive to an oral command.

26. The computer readable medium of claim 23, wherein the process of establishing settings of the apparatus includes one or both of initially setting up the apparatus and optimizing settings of the apparatus.

27. The computer readable medium of claim 23, wherein the processor includes a trained machine learning engine and establishes settings of the apparatus based on outputs of the trained machine learning engine.

28. The computer readable medium of claim 27, wherein the trained machine learning engine is configured to determine an organ (i), a type of examination (ii), a use case of a particular received image (iii), or any combination of (i), (ii) or (iii), and the outputs of the trained machine learning engine include presets of an ultrasonic transducer frequency (iv), an ultrasonic transducer gain (v), and signal processing filter parameters (vi), or any combination of (iv), (v) or (vi).

29. The computer readable medium of claim 27, wherein the trained machine learning engine is configured to make a comparison of parameters from the received data to parameters established by a training data set and establish the settings based on the comparison.

30. The computer readable medium of claim 23, wherein the machine learning process includes training based on preferences expressed by an individual operator.
